(19) United States
(12) Patent Application Publication
(10) Pub. No.: US 2026/0256345 A1
(43) Pub. Date: Sep. 3, 2026

(54) MEDICAL DEVICE INSPECTION SCOPE

(71) Applicant: Clarus Medical, LLC, Minneapolis, MN (US)

(72) Inventors: Thomas Donald Chandler, Coon Rapids, MN (US); Steven Edward Lombardi, New Brighton, MN (US); Mark Francis Brown, Minnetonka, MN (US); Justin Andrew Hawley, Minneapolis, MN (US); Jerome Steven Stepanek, Eden Prairie, MN (US)

(73) Assignee: Clarus Medical, LLC, Minneapolis, MN (US)

(21) Appl. No.: 19/238,086

(22) Filed: Jun. 13, 2025

Related U.S. Application Data
(63) Continuation of application No. 17/591,793, filed on Feb. 3, 2022, now Pat. No. 12,357,164.
(60) Provisional application No. 63/145,066, filed on Feb. 3, 2021.

(51) Int. Cl. A61B 1/05; A61B 1/00; A61B 1/005; A61B 1/06

(52) U.S. Cl. CPC A61B 1/05; A61B 1/00043; A61B 1/00114; A61B 1/00167; A61B 1/005; A61B 1/06; A61B 1/00059; A61B 1/00124; A61B 2562/0223

(57) ABSTRACT

A medical device inspection system includes a console and a flexible imaging scope. The console includes a housing, a video processor, a light source, a visual display on the housing, and an outlet with two ports. The flexible imaging scope is removably connected to the console via the outlet and includes a CMOS image sensor, an optical fiber bundle, and a connector assembly mounted to a proximal end of the imaging scope. The connector assembly includes an electrical image connector and a light source connector, which plug into the two ports of the outlet to connect the flexible imaging scope to the console.

### CROSS-REFERENCE TO RELATED APPLICATIONS

This application is a continuation of U.S. patent application Ser. No. 17/591,793, filed on Feb. 3, 2022, entitled, MEDICAL DEVICE INSPECTION SCOPE, which claims the benefit of U.S. Provisional Patent Application No. 63/145,066, filed on Feb. 3, 2021, entitled, MEDICAL DEVICE INSPECTION SCOPE, the disclosures of which are hereby incorporated by reference in their entireties. To the extent appropriate, a claim of priority is made to each of the above-disclosed applications.

### TECHNICAL FIELD

This application is directed to medical devices, systems and methods. More specifically, the application is directed to a scope for facilitating the inspection of medical devices.

### BACKGROUND

Millions of medical devices are used in hospitals throughout the world every day. With the continuing advancement of medical and surgical procedures over time, one of the trends is toward minimally invasive procedures performed through smaller incisions or even through the body's natural orifices. Examples of this trend include arthroscopic surgery, transcatheter aortic valve replacement (“TAVR”), natural orifice transluminal endoscopic surgery (“NOTES”), robotic surgery and many others. Many of these procedures involve the use of long, flexible catheter instruments and/or long, flexible endoscopes for visualizing the procedure. Additionally, endoscopes are used in countless different diagnostic and therapeutic procedures in many parts of the body.

One of the challenges with the use of endoscopes, fiber scopes, catheter-based medical/surgical instruments and other long, thin, reusable instruments is how to properly and effectively clean them. Many endoscopes and other instruments are too expensive to be disposable and so must be reused. Long, small-diameter, flexible instruments can be extremely hard to clean on the inside. They are also hard to inspect on the inside. Not only can flexible instruments collect bacteria and other contaminants, but they can also crack or become otherwise permanently deformed during use, for example when the instrument is bent or kinked. These instruments are typically processed in a cleaning facility located within the hospital, by workers with very little training. One way to inspect the inside of such instruments is to advance a small, flexible scope through the lumen(s) of the device, so that contaminants and damage can be seen. It can be difficult, however, for the person doing the inspection to effectively identify contaminants and internal damage to the device. Thus, the inspection process can be labor intensive and sometimes ineffective. It can also be hard to find a scope small enough to fit through the lumens of some medical devices while allowing for adequate visualization. Additionally, once contamination of an endoscope or catheter lumen (or similar inner portion of a medical device) is identified, it can often be difficult to adequately clean the lumen.

Therefore, it would be desirable to have improved devices, systems and methods for inspecting and possibly even disinfecting medical devices, specifically endoscopes, catheters and other long, thin, flexible medical devices that are difficult to inspect on the inside. At least some of these objectives are addressed in this application.

### SUMMARY

In one aspect of the present disclosure, a medical device inspection system includes a console and a flexible imaging scope. The console includes a housing, a video processor, a light source, a visual display on the housing, and an outlet with two ports.

The flexible imaging scope is removably connected to the console via the outlet and includes a CMOS image sensor, an optical fiber bundle, and a connector assembly mounted to a proximal end of the imaging scope. The connector assembly includes an electrical image connector and a light source connector, which plug into the two ports of the outlet to connect the flexible imaging scope to the console.

In various embodiments, the console is compatible with at least one additional flexible imaging scope that has at least one different characteristic from the flexible imaging scope. For example, the different characteristic may be image size, scope diameter and/or scope length. In some embodiments, the video processor is configured to process images from multiple different CMOS sensors with different pixel sizes in different flexible imaging scopes.

The connector assembly of the flexible imaging scope may include a permanent magnet, and the console may include a magnetic sensor inside the outlet, to detect the permanent magnet when the connector assembly is plugged into the outlet. The permanent magnet may be configured to indicate to the console a pixel size of the CMOS image sensor. The magnetic sensor may detect the pixel size from the permanent magnet and provide the pixel size to the video processor of the console.

In another aspect of the present disclosure, a method for inspecting a medical device involves plugging a first connector assembly of a first flexible imaging scope into an outlet of a console, where the first connector assembly comprises an electrical image connector and a light source connector. The method then involves identifying a first pixel size of a first image sensor of the first flexible imaging scope with a processor of the console and inspecting the medical device with the first flexible imaging scope. Optionally, the method may further involve unplugging the first flexible imaging scope from the console, plugging a second connector assembly of a second flexible imaging scope into the outlet of the console, identifying a second pixel size of a second image sensor of the second flexible imaging scope with the processor of the console, wherein the first pixel size and the second pixel size are different, and inspecting the medical device with the second flexible imaging scope.

The method may also involve selecting the first flexible imaging scope from a group of multiple different imaging scopes, where at least some of the multiple different imaging scopes have different pixel sizes, and the processor of the console is configured to process images from the multiple different imaging scopes. Identifying the first pixel size may involve sensing a permanent magnet in the connector assembly of the first flexible imaging sensor with a magnetic sensor of the console and providing sensed data from the magnetic sensor to the processor of the console.

These and other aspects and embodiments are described more fully below, in reference to the attached drawing figures.

### BRIEF DESCRIPTION OF THE DRAWINGS

FIG. 1 is a perspective view of a medical device inspection scope, according to one embodiment;

FIG. 2 is a cross-sectional view of the inspection scope of FIG. 1;

FIG. 3A is a perspective view of a medical device inspection system including a console and a flexible imaging scope according to one embodiment; scope, and

FIG. 3B is a side view of a proximal portion of the flexible imaging scope, according to one embodiment; and

FIG. 4 is a side view of four different embodiments of a flexible imaging scope, which are compatible with the console of FIG. 3A.

### DETAILED DESCRIPTION

Disclosed in this application are various examples of an endoscope (or simply “scope”), which may be used for inspecting the inside of medical devices or for any other suitable purpose. For example, the scope may be inserted into a lumen of a larger endoscope and advanced through the lumen to detect imperfections, damage, contamination and/or the like inside of the endoscope. In this way, the scope described in this application may help a user inspect a medical device that is being cleaned, sterilized or otherwise processed for reuse. In some embodiments, the scope may be designed not only to help visualize imperfections and contamination of a medical device lumen but also to disinfect or otherwise clean the lumen. These concepts and many others are described in greater detail below. The examples of various features and embodiments of the scope described below are not intended to limit the scope of the invention but are provided for descriptive purposes only.

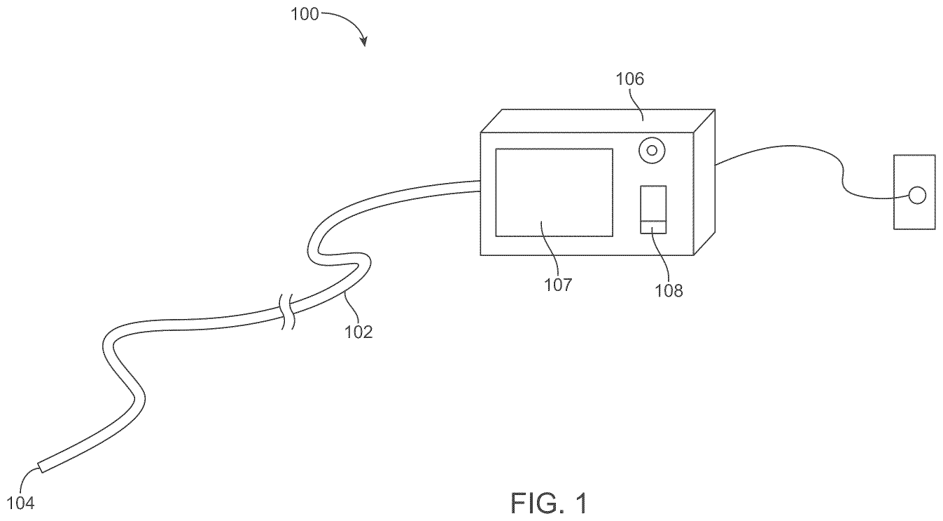

Referring now to FIG. 1, in one example, an elongate, medical device inspection scope 100 includes an outer layer 102 (or “outer housing”) and a distal end 104. The proximal end of the scope 100 is attached to a camera body/light source 106, which may sometimes be referred to as a “box.” In some embodiments, the outer layer 102 of the scope 100 may have a specific diameter, sized to be able to fit within lumens of various endoscopes, catheters and/or other medical devices for inspection purposes. For example, in various embodiments, the outer layer 102 may have an outer diameter of less than 2 millimeters, and in some embodiments less than 1 millimeter.

In one embodiment, the camera body/light source 106 may include a display 107 and one or more controllers 108. The display 107 may display an image of the inside of the medical device being examined, or it may display data related to the inside of the medical device and/or the scope 100. In some embodiments, the camera body/light source 106 may connect to a separate display monitor for displaying images captured by the scope 100. The controllers 108 may include an on/off power switch and any other switch, knob, controller or the like.

In some embodiments, the scope 100 is configured to (1) emit illuminating light and capture still and/or video images of the inside of a medical device and (2) emit cleaning/disinfecting light, such as UV light, UVC light, and/or visible cleaning/disinfecting light (e.g., light having a wavelength from 400 nm to 500 nm, which is blue light that is safe for humans but kills most, if not all, forms of bacteria, yeast, and mold) to clean/disinfect the inside/lumen of the medical device. Such an embodiment may be configured such that the user can switch back and forth between visible/illumination light and cleaning/disinfecting light emission, and/or the user may in some cases emit both types of light simultaneously. In such embodiments, the controllers 108 may include a function control switch, button, knob or the like, for switching back and forth between illumination mode, cleaning/disinfecting mode and in some examples a combination light mode. In one embodiment, the light selection controller 108 may be a mechanical switch that allows the operator to toggle between visible/illumination light and cleaning/disinfecting light. The switch may be rotary or leveler action and may be hand or foot operated. In another embodiment, the controller 108 may be an electronically activated switch, such as an electronic button on the display 107 or another screen, to switch between visible and cleaning/disinfecting light. Embedded software, for example residing in the light source/box 106, may be configured to control the cleaning/disinfecting light energy dwell time (on/off/pulsed—e.g., the amount of energy delivered over a specific time period) and intensity. In one embodiment, a connector (not shown) splices the two different inputs (i.e., the visible light and the cleaning/disinfecting light) into one single output, while preventing the individual light energies from escaping and while withstanding the caustic nature of UVC light (or other disinfecting/cleaning light). In one embodiment, both wavelengths enter a housing through a standard connection point (not shown) and are then spliced into one exit point. In various examples, any suitable splice connector may be used.

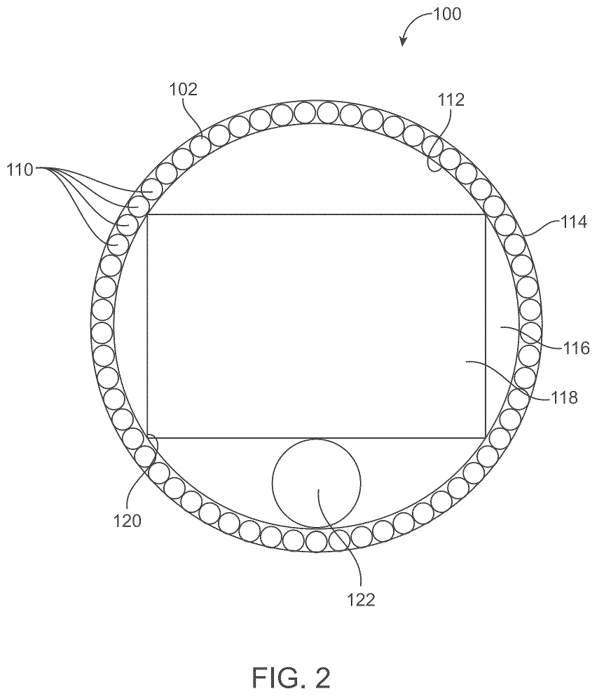

Referring to FIG. 2, the scope 100 of FIG. 1 is illustrated in cross section. As seen in this figure, the scope 100 includes the outer layer 102 and an inner layer 112, which together form a circumferential space 114 between the two. Again, in some embodiments, the outer diameter of the outer layer 102 may be less than 2 millimeters, or in some examples less than 1 millimeter. Multiple light fibers 110 (or “illumination fibers”), which transmit light from the light source 106 to the distal end 104 of the scope 100, may be positioned inside the circumferential space 114 (as illustrated), inside the central lumen 116, or both. Inside the inner layer 112 is a central lumen 116, which contains a camera module 118 and an optional elongate stiffening member 122. In this embodiment, the camera module 118 has a rectangular cross-sectional shape. In an alternative embodiment, the corners 120 of the camera module may be shaved, sanded or otherwise smoothed or rounded off.

As mentioned above, in some embodiments, the light fibers 110 are configured to emit visible light for illumination purposes and cleaning/disinfecting light, such as UV light, UVC light, and/or visible spectrum blue light for disinfecting the inside of a medical device. As UVC light can be highly caustic, it is important to transmit the light down the length of the light fibers 110 carefully, to prevent its unintended release. In some embodiments, each of the light fibers 110 may include a silica core, a doped silica clad, a polyimide layer, and a buffer made of polyimide, silicone, acrylate, fluoropolymer or other suitable buffer material. These are merely examples, however, and in alternative embodiments other materials or combinations may be used. In some embodiments, all the light fibers 110 may be configured to transmit visible light and UV light. In alternative embodiments, one set of fibers 110 may be configured to transmit visible light, and another set of fibers 110 may be configured to transmit UV light.

The stiffening member 122 is an optional component, used in some embodiments to enhance/increase the rigidity of the scope 100, to prevent over-bending or kinking. The stiffening member 122 may have any suitable size, length, shape and material, according to various embodiments. In some embodiments, for example, the stiffening member 122 may be a fiber, such as a glass or plastic fiber with a coating. In one embodiment, the stiffening member 122 may be a laser fiber, which in the scope 100 is not used for transmitting light but only as a piece to add rigidity to the scope 100.

In one embodiment, a method for making the medical device inspection scope 100 may involve positioning the inner layer 112 inside the outer layer 102, then placing the multiple light fibers 110 inside the circumferential space 114. The camera module 116 and laser fiber 122 may then be placed in the inner lumen 116 of the scope 100. In one example, the method of making the scope 100 may also include shaving, sanding or otherwise smoothing off corners 120 of the camera module 118.

A method for using the medical device inspection scope 100 may involve inserting the distal end 104 of the scope 100 into a lumen of a medical device, such as an endoscope, catheter or any other suitable device. The scope 100 is then advanced through the lumen, while the light fibers 110 are used to illuminate the lumen, and the camera module 116 is used to capture video and/or still images of the lumen. Some embodiments may include a processor for storing and/or interpreting data acquired by the camera module. For example, in some embodiments, the processor may be configured to identify irregularities or defects in the inside of an endoscope, catheter or other medical device.

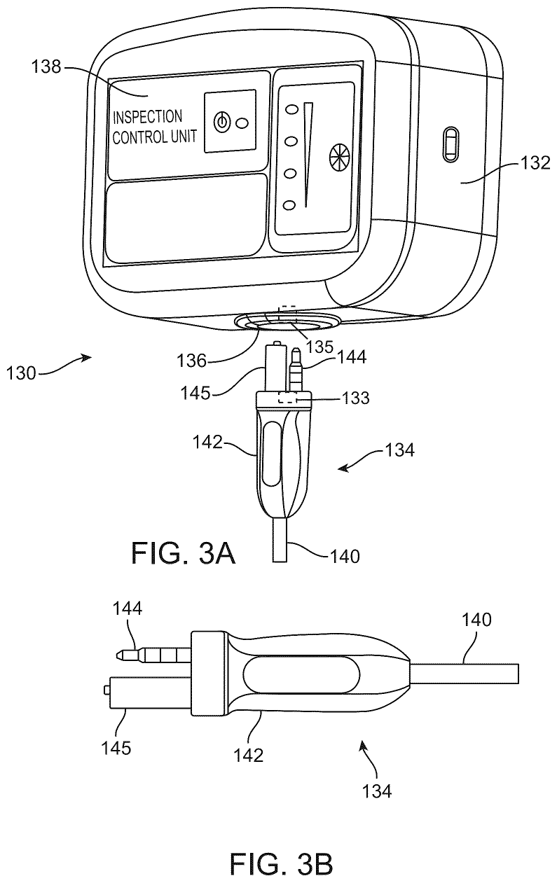

Referring now to FIG. 3A, an imaging scope system 130 is illustrated, according to one embodiment. In this embodiment, the system 130 includes a console 132 (or “controller housing”) and an imaging scope 134. The console 132 includes a display panel 138 and an outlet 136. The imaging scope 134, only a portion of which is shown, includes an imaging cable 140 (which extends to a distal imaging tip, at an opposite end not shown here) attached to a connector assembly 142, which includes an electrical image connector 144 and a light source connector 145. FIG. 3B shows the proximal end of the imaging scope 134 in a slightly larger view. In this embodiment, the imaging scope 134 is removable from the console 132, via the plug 142 and the outlet 136. The electrical image connector 144 and a light source connector 145 plug into two corresponding ports of the outlet 136 to connect the imaging scope 134 to the console 132. This allows the imaging scope 134 to be swapped out for a different imaging scope. Different imaging scopes 134 may have different lengths, diameters, and pixel sizes. For example, the imaging scope 134 may have a 2 mm dimeter, with an image size of 400×400 pixels, or alternatively a 1 mm diameter with an image size of 200×200 pixels. These are merely two examples. The imaging scope 134 includes both an image sensor and an illumination light fiber bundle, and it is terminated in the connector assembly 142 with the electrical image connector 144 and the light source connector 145. In this embodiment of the imaging scope 134, the electrical image connector 144 and the light source connector 145 are arranged side-by-side on the connector assembly 142. In alternative embodiments, however, other configurations may be used. For example, the electrical image connector 144 and the light source connector 145 may be coaxially arranged, with the image fiber bundle within the center pin of a stereo plug.

The console 132 may include an adjustable light source and a video processor that is electrically switchable to process images from either a 400×400 pixel sensor or a 200×200 sensor, for example. In some embodiments, a permanent magnet 133 may be incorporated into the connector assembly 142 of the image scope 134, and the mating outlet 136 in the console 132 may contain a magnetic switch 135 to sense which imaging scope is plugged in. The video processor may then be automatically configured, based on the magnetic signal input.

Previously described systems do not include an imaging scope that combines light and image signal into one connector. They also do not include a video processor that can handle multiple sizes of image sensors or a magnetic sensor built into a connector to indicate to the processor which image sensor is plugged in.

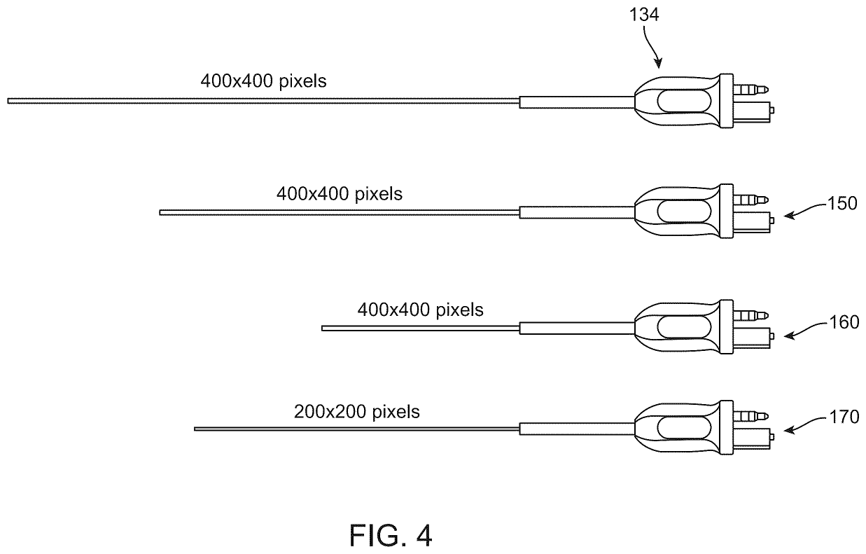

FIG. 4 is a side view of the imaging sensor 134 and several alternative embodiments of imaging sensors 150, 160, 170, which may be used interchangeably with the console 132. As indicated in FIG. 4, the imaging sensor 134 has an image size of 400×400 pixels and is 1.8 m long. Another imaging sensor 150 may have an image size of 400×400 pixels and be 1.1 m long. Another imaging sensor 160 may have an image size of 400×400 pixels and be 0.7 m long. And yet another imaging sensor 170 may have an image size of 200×200 pixels and be 1.0 m long. In various embodiments, imaging sensors for use with the console 132 may have any suitable diameter, length and image size, including but not limited to those shown in FIG. 4.

The above description is intended to be a complete description of one embodiment of a small diameter endoscope for inspecting medical devices and potentially other uses. It is meant to be a description of examples only and is not intended to limit the scope of the invention.

1-10. (canceled)

11. An imaging scope configured to be removably connected to a console, the imaging scope comprising:
a connector arranged at a proximal end of the imaging scope and comprising:
a connector body having a handle portion and a first face;
an electrical image connector prong that extends perpendicularly from the first face of the connector body; and
a light source connector prong that extends perpendicularly from the first face of the connector body and is spaced apart from the electrical image connector prong, the light source connector prong being configured to receive light originating from a light source of the console;
an image sensor arranged at a distal end of the imaging scope; and
an elongate flexible body arranged between the connector and the distal end of the imaging scope, the elongate flexible body comprising:
at least one light fiber within the elongate flexible body that transmits the light received by the light source connector prong toward the distal end of the imaging scope; and
at least one electrical conductor within the elongate flexible body that conducts electrical signals between the image sensor and the electrical image connector prong.

12. The imaging scope of claim 11, wherein the electrical image connector prong is a stereo audio-type prong.

13. The imaging scope of claim 11, wherein the connector is the only connector of the imaging scope that connects to the console.

14. The imaging scope of claim 11, wherein the light source connector prong is a fiber optic connector.

15. The imaging scope of claim 11, wherein the elongate flexible body has an outer diameter of less than 2 millimeters.

16. The imaging scope of claim 11, wherein the elongate flexible body further comprises a stiffening member configured to resist over-bending.

17. The imaging scope of claim 11, wherein the connector comprises exactly two connector prongs extending from the connector body, and wherein the electrical image connector prong and the light source connector prong are the only connector prongs of the imaging scope.

18. The imaging scope of claim 11, wherein the electrical image connector prong and the light source connector prong both extend from the same first face of the connector body, and wherein the connector body comprises a single, unitary structure.

19. The imaging scope of claim 11, wherein the connector is a single connector, and wherein the single connector combines both light and image signal transmission, and wherein the electrical image connector prong and the light source connector prong are arranged on the same connector assembly.

20. An imaging system comprising:
an imaging scope configured to be removably connected to a console, the imaging scope comprising:
a connector arranged at a proximal end of the imaging scope and comprising:
a connector body having a handle portion and a first face;
an electrical image connector prong that extends perpendicularly from the first face of the connector body; and
a light source connector prong that extends perpendicularly from the first face of the connector body and is spaced apart from the electrical image connector prong, the light source connector prong being configured to receive light originating from a light source of the console;
an image sensor arranged at a distal end of the imaging scope; and
an elongate flexible body arranged between the connector and the distal end of the imaging scope, the elongate flexible body comprising:
at least one light fiber within the elongate flexible body that transmits the light received by the light source connector prong toward the distal end of the imaging scope; and
at least one electrical conductor within the elongate flexible body that conducts electrical signals between the image sensor and the electrical image connector prong.

21. The imaging system of claim 20, wherein the at least one light fiber is configured to transmit light having wavelengths between 400 nm to 500 nm.

22. The imaging system of claim 20, further comprising a magnet within the connector body and adjacent the first face.

23. The imaging system of claim 20, further comprising a console having an outlet configured to receive the connector of the imaging scope.

24. The imaging system of claim 23, further comprising a magnetic sensor arranged adjacent to the outlet and configured to detect whether a magnet is present within the connector body when the connector is plugged into the outlet.

25. The imaging system of claim 24, wherein the magnetic sensor detects the magnet and sends a signal to the console, and wherein the console determines a pixel size associated with the image sensor based on the signal.

26. The imaging system of claim 20, further comprising the console, wherein the console includes a light source that generates light to be delivered to the at least one light fiber within the elongate flexible body, wherein the light source includes both a visible light source and a UV light source.

27. The imaging system of claim 26, wherein the UV light source comprises a UVC light source.

28. A console configured to be removably connected to an imaging scope, the console comprising:
a housing;
a video processor disposed within the housing;
a light source disposed within the housing; and
an outlet provided at the housing and configured to receive a connector of an imaging scope, the outlet comprising:
a first face of the outlet;
an electrical image port extending perpendicularly inward from the first face of the outlet and being configured to receive an electrical image connector prong of the connector of the imaging scope, and being electrically coupled to deliver electrical signals from the imaging scope to the video processor; and
a light source connector port extending perpendicularly inward from the first face of the outlet and being configured to receive a light source connector prong of the connector and to provide light from the light source to the imaging scope.

29. The console of claim 28, further comprising a display device wherein the console displays images captured by the imaging scope on the display device.

30. The console of claim 28, wherein the electrical image port is configured to receive a stereo audio-type prong of the connector of the imaging scope.

31. An imaging scope configured to be removably connected to a console, the imaging scope comprising:
a connector arranged at a proximal end of the imaging scope and comprising:
a connector body having a handle portion and a first face; and
a first prong that extends perpendicularly from the first face of the connector body, the first prong comprising a stereo audio-type prong;
an image sensor arranged at a distal end of the imaging scope; and
an elongate flexible body arranged between the connector and the distal end of the imaging scope, the elongate flexible body comprising:
at least one electrical conductor within the elongate flexible body that conducts electrical signals between the image sensor and the first prong.

32. The imaging scope of claim 31, wherein the connector further comprises:
a second prong that extends perpendicularly from the first face of the connector body and is spaced apart from the first prong.
